(19) United States
(12) Patent Application Publication
(10) Pub. No.: US 2015/0037276 A1
(43) Pub. Date: Feb. 5, 2015

(54) HYDROCOLLOID COMPOSITION AND AN ARTICLE CONTAINING THE SAME

(71) Applicants: Ting Fan, Shanghai (CN); Ying Wei Xie, Shanghai (CN); Jing Huang, Shanghai (CN); Kai Qiu, Shanghai (CN); Grace Ho, Shanghai (CN); Dong Wu, Shanghai (CN); Jie Hu, Shanghai (CN)

(72) Inventors: Ting Fan, Shanghai (CN); Ying Wei Xie, Shanghai (CN); Jing Huang, Shanghai (CN); Kai Qiu, Shanghai (CN); Grace Ho, Shanghai (CN); Dong Wu, Shanghai (CN); Jie Hu, Shanghai (CN)

(73) Assignee: 3M INNOVATIVE PROPERTIES COMPANY, SAINT PAUL, MN (US)

(21) Appl. No.: 14/355,916

(22) PCT Filed: Nov. 8, 2011
(86) PCT No.: PCT/CN2011/081917

§ 371 Date: Sep. 12, 2014

(51) Int. Cl. A61K 9/70; A61K 36/23; A61K 31/045; A61K 36/53; A61L 26/00; A61K 31/785; A61K 31/015; A61K 36/61

(52) U.S. Cl. CPC A61K 9/7023; A61K 31/015; A61K 36/23; A61K 36/61; A61K 36/53; A61L 26/0061; A61K 31/785; A61L 26/0014; A61K 31/045
USPC 424/78.02; 514/763; 424/725; 424/745; 514/729

(57) ABSTRACT

The present invention provides a hydrocolloid composition which, based on 100% by weight of the hydrocolloid composition, comprises: 10-90%) by weight of a polyisobutylene tackifier; 5-55% by weight of a hydrophilic absorbing substance; and 0.1-20% by weight of a functional ingredient. The invention further provides an article containing the hydrocolloid composition.

### TECHNICAL FIELD

The present invention relates to a hydrocolloid composition and an article containing the same, and specifically, to a functional hydrocolloid composition used for personal care or medical treatment and an article containing the same.

### BACKGROUND ART

Products containing natural plant ingredients are widely used in personal care or medical treatment and exhibit different efficacies, for example, mind-refreshment, mosquito-repellence, anti-acne, anti-itch or the like. The typical products are essential balm, cooling ointment, perfume or the like. Besides these traditional products, there are also some natural plant products of adhesive plaster types or hydrogel types in the market. However, all of the above products have their respective disadvantages.

Liquid-Type or Ointment-Type Products

Generally, they are applied to respective portions on the skins by hands, for example, a product for mind-refreshment can be applied to temples or the like. As to this application method, firstly, these products will contaminate fingers and clothes, and a portion of the products will be wasted during the application. Some products will be removed by outside friction. Additionally, these products volatilize easily after being applied to an affected part and the lasting time thereof is short. If there are some skin damages in the affected part, the products will cause local stimulation and pain to the skin.

Traditional Adhesive Plaster-Type Products:

Most of the traditional adhesive plasters are based on small molecular materials. The most important problem with these products is that the adhesive thereof will irritate the skin. Furthermore, these products have poor air permeability and large residual adhesive amount, which will cause itch and inflammation to skin. Some of the plasters will stain the skin due to adhesive residues.

Gel-Type Products:

They have the disadvantages of lower strength and poor integrity.

Therefore, in order to realize the efficacy of natural plants, it is still demanded for a composition or an article thereof, which not only has better strength and integrity, but also can avoid the disadvantages of the above products.

Contents of Invention

Therefore, in the first aspect of the invention, it provides a hydrocolloid composition which, based on 100% by weight of the hydrocolloid composition, comprises:
10-90% by weight of a polyisobutylene tackifier;
5-55% by weight of a hydrophilic absorbing substance; and
0.1-20% by weight of a functional ingredient.

Preferably, the hydrocolloid composition further comprises 0.1-50% by weight of a hydrophobic unsaturated elastomeric homopolymer, based on 100% by weight of the hydrocolloid composition.

Preferably, the hydrocolloid composition further comprises 0.1-55% by weight of a resin tackifer, based on 100% by weight of the hydrocolloid composition.

Preferably, the hydrocolloid composition further comprises 0.1-20% by weight of a penetration facilitator, based on 100% by weight of the hydrocolloid composition.

In the second aspect of the invention, it provides an article containing the hydrocolloid composition in the first aspect of the invention.

The hydrocolloid composition of the invention has sufficient strength and integrity, and can provide various functions as demanded. For example, the hydrocolloid composition of the invention can be used for wound healing, or whelk care (reducing the infection from propionibacterium acnes), or mind-refreshment, or anti-itch, or anti-acnes, or mosquito-repellence, or antimicrobial or the like.

### SPECIFIC MODE FOR CARRYING OUT THE INVENTION

The present invention relates to a gelling agent (a hydrophilic substance which can form a colloid upon being contacted with water). Because the gelling agent will form an amorphous colloid after being contacted with water, it is necessary for it to be used in combination with other materials with better strength. In the invention, unless specifically stated otherwise, the term “hydrocolloid” means this composition containing a gelling agent.

The present invention provides a hydrocolloid composition which, based on 100% by weight of the hydrocolloid composition, comprises:
10-90% by weight of a polyisobutylene tackifier;
5-55% by weight of a hydrophilic absorbing substance;
0.1-20% by weight of a functional ingredient.

Preferably, the hydrocolloid composition further comprises 0.1-50% by weight of a hydrophobic unsaturated elastomeric homopolymer, based on 100% by weight of the hydrocolloid composition.

Preferably, the hydrocolloid composition further comprises 0.1-55% by weight of a resin tackifer, based on 100% by weight of the hydrocolloid composition.

Preferably, the hydrocolloid composition further comprises 0.1-20% by weight of a penetration facilitator, based on 100% by weight oft the hydrocolloid composition.

The polyisobutylene tackifier comprises 10-90% by weight, preferably 20-85% by weight, more preferably 25-80% by weight, and most preferably 30-75% by weight of the hydrocolloid composition. Preferably, the polyisobutylene tackifier has a weight average molecular weight of 20,000-150,000 and a polydispersity index (PDI) of 1-10. The examples thereof include, but not limited to, SDG-8650 from Shunda, Hangzhou, LM-MH from Exxon, Oppanol B-12 SFN from BASF, as well as PIB 6H from RitChem, wherein SDG-8650 from Shunda, Hangzhou is preferable, Oppanol B-12 SFN from BASF is more preferable, and PIB 6H from RitChem is most preferable.

The absorbing substance comprises 5-55% by weight, preferably 5-50% by weight, more preferably 7-45% by weight, and most preferably 10-40% by weight of the total weight of the hydrocolloid composition. The absorbing substance means a polymer which can absorb a substance with a weight several times larger than the weight itself of the polymer. The examples thereof include, but not limited to, celluloses, such as HSI00000YP2 hydroxyethyl cellulose from Clariant Chemical, FH5000 carboxymethyl cellulose sodium from Wei Yi, Suzhou, China, Sodium croscarmellose sodium from FMC company; starches such as a carboxymethyl starch from Fuhua, Henan; synthetic resins such as Luquasorb®1030 super water-absorbing polymer from BASF; or the mixtures of the above ingredients, wherein FH5000 carboxymethyl cellulose sodium from Wei Yi, Suzhou is preferable, the combination of FH5000 carboxymethyl cellulose sodium from Wei Yi, Suzhou and the Sodium croscarmellose sodium from FMC company is more preferable, and the combination of the Sodium croscarmellose sodium from FMC company and Luquasorb®1030 super water-absorbing polymer from BASF is most preferable.

The functional ingredient comprises 0.1-20% by weight of the total weight of the hydrocolloid composition, and the addition ratio by weight is different respect to different functions. The functional ingredient means an ingredient which can provide one of the following functions: accelerating wound healing, whelk care (reducing the infection from propionibacterium acnes), mind-refreshment, anti-itch, anti-acnes, mosquito-repellence, antimicrobial and the like. The examples thereof include, but not limited to, limonene, Asiatic Pennywort Herb, tea tree oil, spike essential oil, and water absorbent.

The hydrophobic unsaturated elastomeric homopolymer comprises 0.1-50% by weight, preferably 10-45% by weight, more preferably 15-40% by weight, and most preferably 30-37% by weight of the total weight of the hydrocolloid composition. The hydrophobic unsaturated elastomeric homopolymer is an elastic modified molecular material with a hydrophobic group and an unsaturated double bond, which is formed by polymerization of a monomer. The examples thereof include, but not limited to, polyisoprene (such as, Natsyn 2210 from Goodyear, USA; IR2200 from Zeon, Japan), polybutadiene (such as, BR9000 polybutadiene from Qilu Petrochemical, Sinopec), wherein BR9000 polybutadiene from Qilu Petrochemical is preferable, Natsyn 2210 from Goodyear, USA is more preferable, and IR2200 from Zeon, Japan is most preferable.

The resin tackifer comprises 0.1-55% by weight, preferably 0.1-40% by weight, more preferably 5-30% by weight, and most preferably 5-20% by weight of the total weight of the hydrocolloid composition. The resin tackifer refers to a resin which can increase the viscosity of a rubber. The examples thereof include, but not limited to, Eastotac H-100R Resin from Eastman Chemical, Wingtack 95 from Cray Valley, and Sylvalite RE80HP Rosin Ester from Arizona Chemicals, wherein Eastotac H-100R Resin from Eastman Chemical is preferable, Sylvalite RE80HP Rosin Ester from Arizona Chemicals is more preferable, and Wingtack 95 from Cray Valley is most preferable. Preferably, the resin tackifer has a weight average molecular weight of 200-3000 and a polydispersity index of 0.5-20.

The penetration facilitator comprises 0.1-20% by weight, preferably 0.2-15% by weight, more preferably 0.3-10% by weight, most preferably 0.5-8% by weight of the total weight of the hydrocolloid composition. The penetration facilitator, also referred to as skin-penetrating agent, penetration accelerator, is mainly used to facilitate the penetration of functional ingredients into skin. The examples thereof include, but not limited to, borneol from Huaxin, Jiangxi, China; natural menthol from Jubang Anhui, China; piperine from Sabinsa Company, USA, wherein the borneol from Huaxin, Jiangxi is preferable, the natural menthol from Jubang Anhui is more preferable, and piperine from Sabinsa Company, USA is most preferable.

The hydrocolloid composition of the invention can be prepared simply by the traditional mixing methods.

The invention further provides an article containing the hydrocolloid composition as described above.

The hydrocolloid composition of the invention can be used in various applications. For example, it can be used in the products for mind-refreshment, such as a patch attached to head in the case of examination, meeting, driving, and working. It can further used for anti-itch and wound healing. In summer, the bite from mosquitoes will cause itch and inflammation to skin, and sometimes, it even results in a low-grade wound or skin ulceration. An anti-itching hydrocolloid patch prepared from the hydrocolloid composition of the invention has the following functions of: persistently releasing anti-itching ingredients; absorbing filtering liquid from the wound; forming an excellent barrier on the wound; protecting the bared wound from bacteria invasion; and accelerating wound healing. The hydrocolloid composition of the invention can further be used to protect heels. Heels are liable to be hurt by shoes, specifically, high-heel shoes or new shoes, which often result in skin damages, blisters on feet or the like. As to a hydrocolloid heel patch made of the hydrocolloid composition of the invention added with a functional substance having relaxing efficacy, it can relax the foot and remove fatigue, and additionally, the soft hydrocolloid material can alleviate pressure on the foot and isolate sharp portions on a shoe from the foot. In the case of skin damages, it can also protect the bared wound and accelerate wound healing. The hydrocolloid composition of the invention can further made into an antimicrobial hydrocolloid dressing.

The invention is described in more detail with reference to the examples. It should be noted that these examples are illustrative and do not limit the invention in any way. Unless specifically stated otherwise, the percentages, contents, proportions or the like in the invention are all in terms of weight, and the temperature used in the invention refers to degree centigrade.

### EXAMPLE

The sources of the raw materials used in the following examples are listed in the following Table 1.

TABLE 1
Sources of the raw materials used in Examples

| | Ingredients | Sources |
|---|---|---|
| Example 1 | BR9000 polybutadiene | Qilu Petrochemical |
| | PIB 6H polyisobutylene | RitChem |
| | HS100000YP2 hydroxyethyl cellulose | Eastman Chemical |
| | Borneol | Huaxin, Jiangxi |
| | Limonene | Sinopharm |
| Example 2 | Natsyn 2210 polyisoprene | Goodyear |
| | SDG-8650 polyisobutylene | Shunda, Hangzhou |
| | FH5000 carboxymethyl cellulose | Wei Yi, Suzhou |
| | Asiatic Pennywort Herb | Elgin |
| | Tea tree oil | Elgin |
| | Menthol | Jvbang, Anhui |
| Example 3 | IR2200 polyisoprene | Zeon, Japan |
| | Oppanol B-12 SFN polyisobutylene | BASF |
| | Wingtack 95 | Cray Valley |
| | Sodium croscarmellose, | FMC |
| | Spike essential oil | Sabinsa |
| | Tea tree oil | Sabinsa |
| | Menthol | Jvbang, Anhui |
| Example 4 | Natsyn 2210 polyisoprene | Goodyear |
| | Oppanol B-12 SFN polyisobutylene | BASF |
| | FH5000 carboxymethyl cellulose | Wei Yi, Suzhou |
| | Sodium croscarmellose | FMC |
| | Luquasorb ®1030 | BASF |
| Example 5 | BR9000 polybutadiene | Qilu Petrochemical |
| | PIB 6H polyisobutylene | RitChem |
| | FH5000 carboxymethyl cellulose | Wei Yi, Suzhou |
| | Sodium croscarmellose | FMC |
| | Wind medicated oil | Shuixian, |
| | | Zhangzhou |
| Example 6 | PIB 6H polyisobutylene | RitChem |
| | FH5000 carboxymethyl cellulose | Wei Yi, Suzhou |
| | Sodium croscarmellose, | FMC |
| | Polyhexamethylene Biguanide (PHMB) | Arch Chemicals |
| | COSMOCIL ™ CQ | |
| | PIB 6H polyisobutylene | RitChem |

### Example 1

### Mind-Refreshing Patch

32.5% of BR9000 polybutadiene, 40% of PIB 6H polyisobutylene, 22% of HS100000YP2 hydroxyethyl cellulose, 5% of borneol, and 0.5% of Limonene were added into a mixer, mixed for 2 hr, evacuated and remixed for 1 hr, and then brought it out.

The mixture was weighed and placed between two organosilicon release papers, and then pressed into a sheet with a thickness of 0.05±0.01 mm using a plate curameter. Subsequently, a release paper was peeled from the sheet, and the sheet was then combined with a transparent thin film. The sheet was cut into a certain shape as desired.

### Example 2

### Anti-Itching Patch

50% of Natsyn 2210 polyisoprene, 20% of SDG-8650 polyisobutylene, 22% of FH5000 carboxymethyl cellulose, 5% of Asiatic Pennywort Herb, 2% of Tea tree oil, and 1% of menthol were added into a mixer, mixed for 2 hr, evacuated and remixed for 1 hr, and then brought out.

The mixture was weighed and extruded into a sheet with a thickness of 0.05±0.01 mm using a single screw extruder. The extruded sheet was combined with an organosilicon release paper on the bottom thereof and a transparent thin film on the top thereof, and then wound. The sheet was cut into a certain shape as desired.

### Example 3

### Anti-Acne Patch

23.5% of IR2200 polyisoprene, 35% of Oppanol B-12 SFN polyisobutylene, 15% of Wingtack 95, 20% of the Sodium croscarmellose, 1.5% of Spike essential oil, 2% of Tea tree oil, and 3% of menthol were added into a mixer, mixed for 2 hr, evacuated and remixed for 1 hr, and then brought out.

The mixture was weighed and extruded into a sheet with a thickness of 0.03±0.005 mm using a single screw extruder. The extruded sheet was combined with an organosilicon release paper on the bottom thereof and a transparent thin film on the top thereof, and then wound. The sheet was cut into a certain shape as desired.

### Example 4

### Super Water-Absorbing Hydrocolloid Patch

37.5% of Natsyn 2210 polyisoprene, 35% of Oppanol B-12 SFN polyisobutylene, 8% of FH5000 carboxymethyl cellulose, 6% of the Sodium croscarmellose, and 13.5% of Luquasorb®1030 were added into a mixer, mixed for 2 hr, evacuated and remixed for 1 hr, and then brought out.

The mixture was weighed and extruded into a sheet with a thickness of 0.03±0.005 mm using a single screw extruder. The extruded sheet was combined with an organosilicon release paper on the bottom thereof and a transparent thin film on the top thereof, and then wound. The sheet was cut into a certain shape as desired.

### Example 5

### Mind-Refreshing Patch

32.5% of BR9000 polybutadiene, 40.0% of PIB 6H polyisobutylene, 15.0% of FH5000 carboxymethyl cellulose, 6.5% of sodium croscarmellose, and 6.5% of Wind medicated oil were added into a mixer, mixed for 2 hr, evacuated and remixed for 1 hr, and then brought it out.

The mixture was weighed and placed between two organosilicon release papers, and then pressed into a sheet with a thickness of 0.05±0.01 mm using a plate curameter. Subsequently, a release paper was peeled from the sheet, and the sheet was then combined with a transparent thin film. The sheet was cut into a certain shape as desired.

### Example 6

60% of PIB 6H polyisobutylene, 15.0% of FH5000 carboxymethyl cellulose, 9.5% of sodium croscarmellose, and 5% Polyhexamethylene Biguanide (PHMB) COSMOCIL™ CQ were added into a mixer, mixed for 2 hr, evacuated and remixed for 1 hr, and then brought it out.

The mixture was weighed and extruded into a sheet with a thickness of 0.03±0.005 mm using a single screw extruder. The extruded sheet was combined with an organosilicon release paper on the bottom thereof and a transparent thin film on the top thereof, and then wound. The sheet was cut into a certain shape as desired.

### Example 7

One patch made in Example 1 was taken. Its weight was 0.2193 g (the same size of backing was weighed 0.0116 g, so hydrocolloid had a weight of 0.2077 g). The patch was soaked in 20 g (approximately 25 mL) ethanol. It was stirred by using magnetic stirrer for 24 h to obtain an extract solution. Then the extract solution was tested via GC-MS.

### Chromatographic Condition:

Column: DB-1 30 m*0.32 mm*1 μm;

Injector temp.=250° C.;

Split ratio=10:1;

Oven:
Initial temp.=120° C.,hold 1 min;
Ramp=5° C./min;
Final temp.=135° C.,hold 5 min.

Carrier gas: N2 (2 ml/min);

Detector: FID; temp.=250° C.

### Standard Preparation:

0.0104 g menthol standard was weighed into 10 mL volumetric flask, dissolved with anhydrous ethanol, and diluted to mark.

### Test Results:

Menthol concentration in the extract solution was 0.0727 mg/mL. Its percentage in the patch was 0.88%. Compared with the dosage 5%, 17.5% menthol was released from the patch.

### Example 8

One patch made in Example 2 was taken. Its weight was 0.2403 g (the same size of backing weighed 0.0116 g, so hydrocolloid had a weight of 0.2287 g). The patch was soaked in 20 g (approximately 25 mL) ethanol. It was stirred by using magnetic stirrer for 24 h to obtain an extract solution. Then the extract solution was tested via GC-MS.

### Chromatographic Condition:

Column: DB-1 30m*0.32 mm*1 μm;

Injector temp.=250° C.;

Split ratio=10:1;

Oven:
Initial temp.=120° C.,hold 1 min;
Ramp=5° C./min;
Final temp.=135° C.,hold 5 min.

Carrier gas: N2 (2 ml/min);

Detector: FID; temp.=250° C.

Standard Preparation:

0.0261 g borneol standard was weighed into 25 mL volumetric flask, dissolved with anhydrous ethanol, and diluted to mark.

### Test Results:

Menthol concentration in extract solution was 0.0213 mg/mL. Its percentage in the patch is 0.23%. Compared with the dosage 1%, 23.3% borneol was released from the patch.

Examples 5 and 6 proved that the functional ingredients were indeed incorporated into the hydrocolloid matrix.

### Example 9

This example was used to show whether it works by the in house panel test. 13 volunteers attended this test. They were asked to wear the patches made in Example 5 and asked if they had feelings after 5, 10, 30 & 60 min. The results were listed in Table 2.

TABLE 2
Volunteers answers on whether they have feelings after different times

| Volunteer No. | 5 min | 10 min | 30 min | 60 min |
|---|---|---|---|---|
| 1 | Yes | Yes | Yes | No |
| 2 | Yes | Yes | Yes | No |
| 3 | Yes | Yes | Yes | No |
| 4 | Yes | Yes | Yes | Yes |
| 5 | Yes | Yes | Yes | No |
| 6 | Yes | Yes | Yes | No |
| 7 | Yes | Yes | Yes | No |
| 8 | Yes | Yes | Yes | No |
| 9 | Yes | Yes | Yes | No |
| 10 | Yes | Yes | Yes | No |
| 11 | Yes | Yes | Yes | Yes |
| 12 | Yes | Yes | Yes | No |
| 13 | Yes | Yes | Yes | No |

According to the results, 100% of them can still feel the patch after 30 min, even 2 of them still have feelings after 60 min, which is much longer than daubing Wind medicated oil on skin directly.

### Example 10

One patch made in Example 6 was taken. Zone of Inhibition (ZOI) toward Staphyloccocus aureus was tested according to Disinfection Technical Guidelines 2002. The diameter of ZOI is 4.5 cm, which shows strong antimicrobial effectiveness on Staphyloccocus aureus.

1-18. (canceled)

19. A hydrocolloid composition which, based on 100% by weight of the hydrocolloid composition, comprises:
10-90% by weight of a polyisobutylene tackifier;
5-55% by weight of a hydrophilic absorbing substance; and
0.1-20% by weight of a functional ingredient.

20. The hydrocolloid composition according to claim 19, wherein the polyisobutylene tackifier has a weight average molecular weight of 20000-150000 and a polydispersity index of 1-10.

21. The hydrocolloid composition according to claim 19, wherein hydrocolloid composition comprises 30-75% by weight of the polyisobutylene tackifier, based on 100% by weight of the hydrocolloid composition.

22. The hydrocolloid composition according to claim 19, wherein the hydrophilic absorbing substance is selected from the group consisting of celluloses, starches, synthetic resins, and the mixtures thereof.

23. The hydrocolloid composition according to claim 19, wherein hydrocolloid composition comprises 10-40% by weight of the hydrophilic absorbing substance, based on 100% by weight of the hydrocolloid composition.

24. The hydrocolloid composition according to claim 19, wherein the functional ingredient is selected from the group consisting of limonene, asiatic pennywort herb, tea tree oil, spike essential oil, water absorbent, antimicrobial, wound healing, and the mixtures thereof.

25. The hydrocolloid composition according to claim 19, wherein the hydrocolloid composition fulther comprises 0.1-50% by weight of a hydrophobic unsaturated elastomeric homopolymer, based on 100% by weight of the hydrocolloid composition.

26. The hydrocolloid composition according to claim 24, wherein the hydrophobic unsaturated elastomeric homopolymer is selected from the group consisting of polyisoprene, polybutadiene, and the mixtures thereof.

27. The hydrocolloid composition according to claim 19, wherein the hydrocolloid composition comprises 30-37% by weight of the hydrophobic unsaturated elastomeric homopolymer, based on 100% by weight of the hydrocolloid composition.

28. The hydrocolloid composition according to claim 19, wherein the hydrocolloid composition further comprises 0.1-55% by weight of a resin tackifer, based on 100% by weight of the hydrocolloid composition.

29. The hydrocolloid composition according to claim 28, wherein the resin tackifer has a weight average molecular weight of 200-3000 and a polydispersity index of 0.5-20.

30. The hydrocolloid composition according to claim 28, wherein the hydrocolloid composition comprises 30-75% by weight of the resin tackifer, based on 100% by weight of the hydrocolloid composition.

31. The hydrocolloid composition according to claim 28, wherein the hydrocolloid composition further comprises 0.1-20% by weight of a penetration facilitator, based on 100% by weight of the hydrocolloid composition.

32. The hydrocolloid composition according to claim 31, wherein the penetration facilitator is selected from the group consisting of borneol, menthol, piperine, and the mixtures thereof.

33. The hydrocolloid composition according to claim 1, wherein the hydrocolloid composition comprises 0.5-8% by weight of the penetration facilitator, based on 100% by weight of the hydrocolloid composition.
